(19) United States
(12) Patent Application Publication
(10) Pub. No.: US 2015/0342569 A1
(43) Pub. Date: Dec. 3, 2015

(54) Transparency control for medical diagnostic ultrasound flow imaging

(71) Applicant: Siemens Medical Solutions USA, Inc., Malvern, PA (US)

(72) Inventors: Liang Zhai, Castro Valley, CA (US); Sharleen Marshall, San Ramon, CA (US); Annie Ju, Los Altos Hills, CA (US)

(73) Assignee: Siemens Medical Solutions USA, Inc., Malvern, PA (US)

(21) Appl. No.: 14/292,429

(22) Filed: May 30, 2014

(51) Int. Cl. A61B 8/08; G06T 15/50; A61B 8/06

(52) U.S. Cl. CPC A61B 8/5223; A61B 8/06; A61B 8/488; A61B 8/483; G06T 15/503; G06T 2210/41; G06T 2219/2012

(57) ABSTRACT

Flow information can be overwhelming in medical color flow images, including volume color Doppler imaging and vector flow imaging. The flow of interest can be obscured or concealed by the adjacent flows in the displayed images. To enhance the color flow display and highlight the flow of interest, the transparency or opacity for each pixel or voxel in the flow data is modulated. Rather than or in addition to relying on color Doppler parameters, such as velocity amplitude, energy and variance, the direction of the flow is used to modulate transparency.

### BACKGROUND

The present embodiments relate to flow imaging in ultrasound diagnostic imaging. In particular, flow imaging of specific flow regions with reduced interference from other flow regions is provided.

Volume color Doppler imaging allows clinicians to more accurately evaluate cardiac anatomy and flow hemodynamics as compared to two-dimensional color Doppler. In volume imaging, a user may view flow without making geometric assumptions. However, a flow or structure of interest may be obscured by adjacent structures or flows in the rendered volume. For example, during a transesophageal (TEE) exam, it may be challenging to evaluate a mitral regurgitant jet when the adjacent normal left atrial flow moving in the opposite direction is displayed in front of the flow of interest. This interference occurs for views from the perspective of the transducer. Thus, rendering techniques that can selectively highlight a flow of interest and remove or conceal adjacent flows that obscure visualization are desired.

Thresholding and transparency are two approaches to removing interfering flow information. By simply increasing the low velocity threshold or cutoff frequency of the clutter filter, lower velocity flow signals may be removed from the displayed image. Thus, users may selectively keep the high velocity flow by adjusting the clutter filter settings to improve rendering of the mitral regurgitant jet. The adjacent flows of atrial flow may have a sufficiently low velocity, so are rejected or removed. This thresholding approach may compromise sensitivity in the mitral jet since low flow parts of the flow of interest are also removed.

For the transparency approach, transparency values are assigned to each voxel based on either the Doppler parameters (e.g., variance, velocity or combination of these parameters) or a function of B-mode and color flow data. Transparency computations based on Doppler variance assume the Doppler variance represents the degree of flow disturbance. However, researchers have concluded that Doppler variance is more of an indication of aliasing and indicates little of flow disturbance. Transparency based on velocity has the same problems as the thresholding, resulting in loss of sensitivity. Energy may not sufficiently distinguish between the flow regions of different jets. B-mode information represents tissue, so does not distinguish between flow regions.

### BRIEF SUMMARY

By way of introduction, the preferred embodiments described below include a method, system, instructions, and computer readable media for transparency control in medical ultrasound flow imaging. Rather than or in addition to relying on color flow magnitude, the direction of flow modulates transparency. The transparency is controlled using direction of flow. For example, flow towards a transducer is made more opaque and/or flow away from a transducer is made more transparent. Where the flow region of interest flows in a different direction than an interfering flow region, the direction of flow may be used to modulate transparency for reducing the obstruction caused by the interfering flow region.

In a first aspect, a method is provided for transparency control in medical ultrasound flow imaging. Ultrasound flow data is acquired as voxels representing a volume of a patient. A processor determines a direction of flow for each voxel. The processor sets a transparency for each voxel as a function of the respective direction of the flow. The processor renders an image of the volume of the patient with the ultrasound flow data. The rendering is a function of the transparencies for the voxels.

In a second aspect, a system is provided for transparency control in ultrasound medical imaging. An ultrasound imaging system is configured to scan an internal volume of a patient with a transducer. A processor is configured to modulate transparencies of data responsive to the scan as a function of direction of flow and three-dimensionally render a flow image from the data as a function of the transparencies. A display is operable to display the flow image of the internal volume. The flow image comprises a color image where colors associated with one direction are more opaque than colors associated with another direction.

In a third aspect, a non-transitory computer readable storage medium has stored therein data representing instructions executable by a programmed processor for transparency control in medical imaging. The storage medium includes instructions for performing color Doppler imaging, and setting opacity for the performing of the color Doppler imaging as a function of a direction of flow.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is flow chart diagram of one embodiment of a method for transparency control in ultrasound imaging;

FIG. 2 is an example volume rendering without transparency control, and FIG. 3 is an example volume rendering with transparency control based on flow direction; and

FIG. 4 is a block diagram of one embodiment of an ultrasound system for transparency control in ultrasound imaging.

### DETAILED DESCRIPTION OF THE DRAWINGS AND PRESENTLY PREFERRED EMBODIMENTS

Voxel transparency of the flow data is varied based on the flow direction. The transparency is used in generating an image, such as a three-dimensional rendering. In the image, flow data representing flow in one direction will be more transparent than flow data representing flow in another direction.

Compared to clutter filtering or velocity thresholding, transparency modulation by flow direction may avoid compromising sensitivity. By allowing the flow of interest to stand out from the rest of flows and structures, the users may better evaluate the complete flow distribution from various perspectives, including perspectives with an intervening flow structure.

Opaque and transparent are opposites, so setting opacity may be performed by the inverse setting of transparence and vice versa. Where the level of opacity is used, the level is also one of transparency, and vice versa.

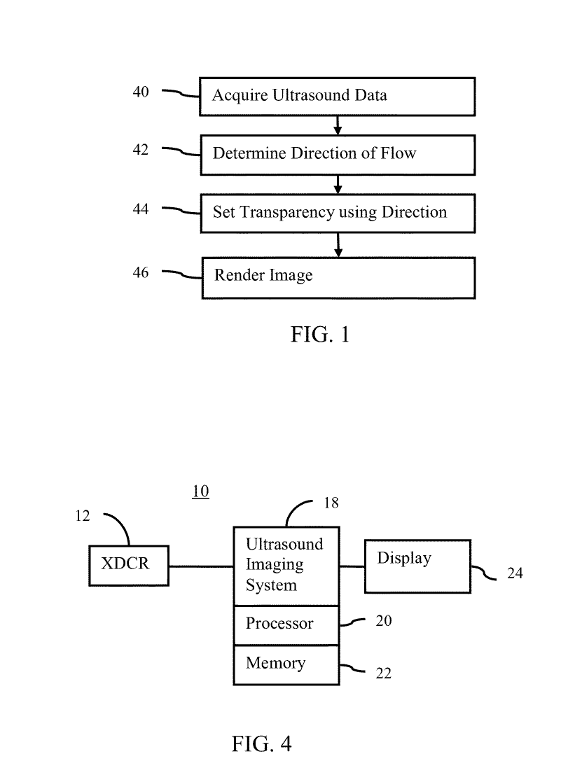

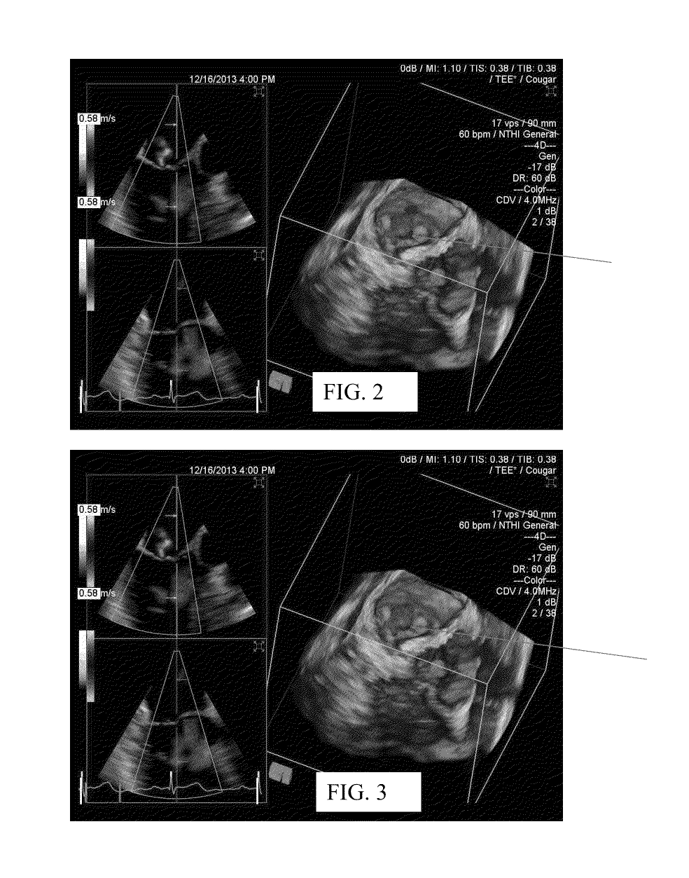

FIG. 1 shows a method for transparency control in medical ultrasound flow imaging. The acts of FIG. 1 are implemented by the system 10 of FIG. 4, a processor, or a different system. For example, data is acquired in act 40 by the processor from memory, transfer from an imaging system, or as part of the data flow path of an imaging system. The processor determines the direction in act 42 using or from the data and sets the transparency in act 44. The processor renders or causes rendering by a graphics processing unit of an image using the transparency in act 46.

The acts shown in FIG. 1 are performed in the order shown or a different order. For example, the data acquisition process, such as Doppler estimation, may provide the direction information in act 42 as part of or before acquisition of the data in act 40.

Additional, different, or fewer acts may be performed. For example, act 46 may not be used. As another example, other acts for rendering, such as selecting a view direction and/or lighting model, are performed. In yet another example, acts for setting or adjusting a clutter filter and/or velocity thresholds as part of rendering or preparing the data for rendering are provided.

The method of FIG. 1 is performed as part of ultrasound flow imaging. For example, color Doppler imaging is provided where the velocity, energy, and/or variance estimates are color mapped. The color of the image represents the estimated flow, with or without B-mode or other tissue information. In one embodiment, the ultrasound flow imaging includes volume rendering, such as volume rendering a Doppler velocity image. In another embodiment, a vector flow display is provided. In vector flow imaging, arrows, streamlines, or other indicators of the flow direction or flow vector are indicated in the image. Both magnitude and direction information are displayed. The ultrasound flow imaging is three-dimensional imaging (e.g., volume rendering where the image is a view of a three-dimensional volume represented by data) or two-dimensional imaging (e.g., planar image representing a two-dimensional area).

The transparency control for the ultrasound flow imaging is performed for each sample contributing to the image. The determination of the direction of flow and setting the transparency are performed separately for each of the locations in the plane or volume. For projection rendering, locations along each ray are composited together, at least until the saturation of the accumulated opacity, using the data as weighted by the transparency. Samples for the entire volume contributing to the rendered image are processed. Transparency may not be set for tissue locations or locations in the volume not associated with a threshold amount of flow (e.g., velocity and/or energy threshold). The processing occurs regardless of whether or not any or what anatomy or flow is represented. The flow may cause a given sample and/or transparency to be different, but segmentation of specific flow regions may not be performed (i.e., not locate or identify specific flow regions from other flow regions in the volume). Alternatively, segmentation and/or clipping is provided.

In act 40, ultrasound data representing a volume or plane of a patient is acquired. In scanning, acoustic energy echoes from the tissue or fluid are received by a transducer. The resulting ultrasound data represents the acoustic echoes from the patient. The scanning may be for B-mode, color flow mode, tissue harmonic mode, contrast agent mode or other now known or later developed ultrasound imaging modes. Combinations of modes may be used, such as scanning for B-mode and Doppler mode data. Any ultrasound scan format may be used, such as a linear, sector, or Vector®. Using beamforming or other processes, data representing the scanned region is acquired. The data is in an acquisition format (e.g., Polar coordinate system) or interpolated to another format, such as a regular three-dimensional grid (e.g., Cartesian coordinate system). Different ultrasound values represent different locations within the volume.

To acquire the data representing a volume of the patient, any type of scanning may be used, such as planar or volume scanning. For planar scanning, multiple planes are sequentially scanned. The transducer array may be rocked, rotated, translated or otherwise moved to scan the different planes from the same acoustic window or multiple acoustic windows. The volume is scanned by electronic, mechanical, or both electronic and mechanical scanning. In one embodiment, many (e.g., 16, 32, 64, or 128) receive beams are formed in response to each broad beam transmission to increase the scan rate for volume scanning. Using any scanning approach, the resulting data represents a volume.

In another embodiment, the ultrasound data is acquired by data transfer or from storage. For example, ultrasound data from a previously performed ultrasound examination is acquired from a picture archival or other data repository. As another example, ultrasound data from an on-going examination or previous examination is transferred over a network from one location to another location, such as from an ultrasound imaging system to a workstation in the same or different facility.

For acquiring flow data by scanning, multiple echoes from the same locations are used. For volume rendering, the complete volume is scanned at different times. Scanning at different times acquires spatial samples associated with flow. Any now known or later developed pulse sequences may be used. A sequence of at least two (flow sample count) transmissions is provided along each scan line. Any pulse repetition frequency, ensemble/flow sample count, and pulse repetition interval may be used. The transmissions along one or more lines may be interleaved with transmissions along one or more other lines. With or without interleaving, the spatial samples for a given time are acquired using transmissions from different times. The samples from different scan lines may be acquired sequentially, but rapidly enough to represent a same time from a user perspective. Multiple scans are performed to acquire samples for different times.

The received spatial samples may be wall filtered/clutter filtered. The clutter filtering is of signals in the pulse sequence for estimating flow at a given time. A given signal may be used for flow estimates representing different times, such as associated with a moving window for clutter filtering and estimation. Different filter outputs are used to estimate flow for a location at different times.

The echo responses to the transmissions of the sequence are used to estimate velocity, energy (power), and/or variance at a given time. Flow data is generated from the clutter filtered samples. Any flow data may be generated, such as velocity, energy (power), and/or variance. Doppler processing, such as autocorrelation, may be used. In other embodiments, temporal correlation may be used. Another process may be used to estimate the flow data. Color Doppler parameter values (e.g., velocity, energy, or variance values) are estimated from the samples acquired at different times. Color is used to distinguish from spectral Doppler imaging, where the power spectrum for a range gate is estimated. The change in frequency between two samples for the same location at different times indicates the velocity. A sequence of more than two samples may be used to estimate the color Doppler parameter values. Estimates are formed for different groupings of received signals, such as completely separate or independent groupings or overlapping groupings. The estimates for each grouping represent the spatial location at a given time. Multiple frames of flow data may be acquired to represent the volume at different times.

The estimation is performed for spatial locations in the volume. For example, velocities for the different planes are estimated from echoes responsive to the scanning. Using data in the acquisition or scan format or data converted to a regular three-dimensional grid, the data represents different locations throughout a volume (e.g., NxMxO distribution of data where N, M, and O are greater than 1). Each location is a sub-volume or voxel. The data is acquired as voxels representing the volume of the patient. Color Doppler data or other flow data is acquired as three-dimensional representation of the energy, velocity, and/or variance of flow where each voxel associated with flow corresponds to an estimate of the flow.

The estimates may be thresholded. Thresholds are applied to the velocities. For example, a low velocity threshold is applied. Velocities below the threshold are removed or set to another value, such as zero. As another example, where the energy is below a threshold, the velocity value for the same spatial location is removed or set to another value, such as zero. Alternatively, the estimated velocities are used without thresholding.

To isolate a flow of interest, the system, processor, or user may alter the settings for the clutter filtering and/or the thresholds. Alternatively, default values are used. Rather than or in addition to thresholding the data used for rendering, the Doppler parameters such as velocity, energy or variance or combinations of these parameters may be used to modulate the amount of change in transparency (e.g., low velocity maps to greater transparency).

In act 42, a direction of flow is determined. The flow of fluid in the patient at each flow location is in a direction. For example, the flow for a jet from a heart valve is, at least at a given point in time, generally in one direction with parts of the flow being in other directions away from the valve. The flow itself for each voxel with flow is a three-dimensional vector.

The direction of the flow for each flow voxel is determined along one, two, or three dimensions. The direction along which flow is determined may have any frame of reference, such as relative to the transducer used to acquire the data or relative to anatomy. In one embodiment, a one-dimensional direction of flow is determined. The direction along the scan lines is used, such as to and away from the transducer. In velocity estimation, the sign of the velocity estimate indicates the motion direction along the scan line relative to the imaging transducer. For example, the velocity is negative if moving away from the transducer and positive if moving towards the transducer. The sign of the estimate may also be switched depending on system implementation, or user preference. While the actual flow may be other than along the scan line, the velocity estimate includes the component of the flow vector that is along the scan line. This direction may be sufficient. The direction of flow for each of a plurality of flow voxels is determined along one or more scan lines in the volume.

In another embodiment, a two or three-dimensional direction of flow is found. Using flow data from different times, the data is correlated while positioned with different spatial offsets. One set of data from one time is translated along one, two, or three dimensions with or without rotation, and correlation is performed for the different amounts and/or directions of translation. The offset with the greatest correlation represents the vector of flow, providing the direction of flow. In other embodiments, the two or three-dimensional direction of flow is found by acquiring flow estimates using different transmit and/or receive apertures spaced apart or centered at different locations on the transducer array. The direction may be estimated from the differences in the estimates relative to the differences in the scan line intersecting the voxel given the offset apertures. Different apertures may be used to find the flow in the azimuth or elevation dimensions. By combining with the magnitude of flow along the scan lines, a two or three dimensional vector is determined. Any now known or later developed approach for measuring the direction of flow along two or more dimensions or a dimension other than the scan line may be used.

In act 44, a transparency is set for each flow voxel. The transparency is set by calculating a transparency from one or more variables, such as determining a transparency from an equation using the sample of the ultrasound data and the direction. Alternatively, the transparency is set by adjusting a previously determined transparency. For example, a transparency is assigned as part of rendering. The transparency is assigned based on the ultrasound flow data, such as providing lower transparency (i.e., higher opacity) for higher values of the ultrasound flow data. Any mapping function may be used. The transparency may be set as unity for rendering or set using any function based on the rendering being performed. Any function, such as multiplication, division, addition, or subtraction, may be used to scale or adjust the transparency. For example, the magnitude of the variance and/or velocity is used to set the transparency. Lighting and/or degree of occlusion may be additionally or alternatively used to set the transparency.

Given the transparency for rendering, the transparency is then further adjusted or set based on the direction of flow. The direction of flow may be the only or just one of the variables used for initially setting or later adjusting the setting of transparency. The transparency may be increased, decreased, maintained the same, or initially set based on the direction of flow.

The transparency is set for each of the flow locations. Since the base transparency value for a given location may be different or the same, the transparency for each sample or voxel may be different or the same. Since the direction of flow for each sample may be different, the transparency for each sample after adjustment may be different or the same. The transparency may be the same or different for different locations. Transparency is varied as a function of the direction of flow throughout an entire volume, or at least for samples contributing to flow in the rendered image.

The transparency is increased or set higher for one direction of flow and is decreased, maintained (e.g., no change or a 1.0 multiplication weight), or set lower for a different direction of flow, such as an opposite direction. Any magnitude of change in transparency may be used. Using a one-dimensional direction of flow, a binary transparency setting or adjustment of the setting is provided. For example, opacity is increased for flow towards a transducer, and opacity is decreased for flow away from the transducer, or vice versa (e.g., setting the transparency to be greater where the direction is towards a transducer and to be lesser where the direction is away from the transducer). In relative terms, the transparency is set to be greater for voxels with a first direction of flow and to be less for a voxels with a second direction of flow different than the first direction. The two directions may or may not be parallel. Alternatively to changing by a certain amount, the transparency is increased or decreased to a certain value. The user may adjust the amount of transparency change or a default is used.

The transparency is set using binary criteria even with two or three-dimensional flow direction information. An axis of interest is selected, such as the view direction or a direction of flow for a particular jet. The component of the direction of flow along that axis is used to determine which of two transparency settings or adjustments to use.

Non-binary transparence based on direction may be used. With a two or three-dimensional direction, further gradations of the transparency based on direction may be provided. For example, the greatest difference in transparency settings are for flows in opposite directions along a direction of the greatest magnitude of flow or along a viewing direction. For voxels with directions at an angle to this axis, the transparency setting or adjustment is less (e.g., less change in transparency). As the direction of flow becomes more orthogonal to the axis, the reduction in opaqueness is less. Any linear, non-linear, or other function may be used to map the adjustment or setting transparency to the direction of flow.

Any transparency value may be set. A coefficient used in rendering or generating an image is set. The transparency for three-dimensional rendering provides relative intensity or brightness for one voxel over another along a viewing direction. For each pixel in a rendered image, multiple voxels along the viewing direction contribute, such as in alpha blending. The transparency weights the contribution, such as setting the alpha value as the transparency for weighting RGB values. For transparency in two-dimensional imaging, there may be no intervening flow. The transparency instead reduces brightness, magnitude, intensity or other characteristics, resulting in flow regions of interest appearing brighter, more intense, or otherwise highlighted as compared to other regions. In both two and three-dimensional imaging, the transparency is used to reduce the visual appearance of undesired flow relative to desired flow, increase the relative visual appearance of desired flow relative to undesired flow, or both. By adjusting transparency as a function of the flow direction, the users may preferentially increase the visibility of the flow of interest.

Flow direction may also be combined with other Doppler parameters to further improve the overall transparency settings in the volume renderer. In addition to setting the transparency as a function of direction, the transparency may be set as a function of the magnitude of the variance, velocity, other Doppler parameter, or combinations thereof. Alternatively or additionally, velocity and/or energy thresholds may be used to identify the flow voxels of interest for rendering with the transparency.

In act 46, an image is generated from the ultrasound data. One or more images are generated from the ultrasound dataset. The image is rendered from the ultrasound data representing the volume. The image is a rendering of the volume. Any type of rendering may be used, such as volume rendering, surface rendering, or other three-dimensional imaging. In alternative embodiments, the image is a two-dimensional image of a plane.

In one embodiment, projection or direct rendering is provided. The projection rendering casts rays through the volume for each pixel in the image. Data along each ray is used to determine the pixel intensity and/or color. Any compositing or projection function may be used, such as averaging, alpha blending, combination, or selection of information (e.g., maximum value selection) from along the viewing direction.

Transparency (e.g., 1-opacity level) is used as part of the rendering. Transparency may be used to differentiate between sections of the volume data for rendering the flow in the patient. The transparency is used to emphasize the data for some locations relative to other locations, such as emphasizing locations with flow in one direction relative to locations with flow in a different direction. The transparency is assigned such that greater transparency is provided for intervening or undesired flow regions, and lesser transparency is provided for voxels showing desired flow.

During compositing, the adjusted transparency and the ultrasound flow data are used. The ultrasound data is a color or scalar value, such as the value mapped to RGB values. The ultrasound data is weighted by the transparency. A greater transparency weights the RGB values to be lower than a lesser transparency. The transparency weighted ultrasound data along the ray line is composited. The compositing may include interpolation from adjacent samples where the ray line is not aligned to the volume or 3D grid.

The depth along each ray line through the volume may be limited. For example, the compositing occurs until a given pixel or composite value reaches a limit, such as the saturation of the accumulated opacity. The accumulated opacity along a ray line may reach a maximum value. Once the maximum accumulated opacity value is reached, the compositing along that ray line is stopped. Ultrasound values and the transparency at deeper depths are not used for the compositing. Starting from a front and proceeding towards a back of the volume from the viewer's direction, the depth at which ultrasound data along a ray line contributes to each pixel may be limited due to saturation of the accumulated opacity.

By adjusting the transparency as a function of the direction of flow, the depth along the ray lines before saturation of the accumulated opacity may be increased. Increasing the transparency increases the depth of the contribution along one or more of the ray lines. For example, a flow region spaced further from a user's view point more likely contributes to the image. Due to the lesser degree of opacity of the intervening flow, the deeper flow may be more easily viewed. The greater transparency results in the ultrasound flow values (e.g., scalar or RGB) of the intervening flow contributing less to the composite value. The transparency adjustment allows colors in an otherwise flow-occluded region to brighten more easily than colors caused by the intervening flow.

Any of the flow data may be used for rendering the image. For example, a velocity image is rendered. The color of the image represents the magnitude of the velocity. Energy or variance may alternatively be used for rendering the image. The magnitude of the velocity, energy, variance or combination thereof is rendered from a volume or voxel dataset to an image for two-dimensional display, where the magnitudes of voxels contributing to each pixel are weighted by the transparency.

In one example, the visualization of a jet is improved. A three-dimensional color Doppler image of valvular flow in the heart of a patient is rendered. Both variance and velocity thresholds are applied. If the variance and velocity of a voxel are both below the thresholds and if the flow goes in the opposite direction of the jet flow of interest, a high transparency value is applied. By doing so, the turbulent jet flow, which is usually associated with a large range of flow velocities and aliasing will be well portrayed and emphasized in the display. The more opaque colors associated with flow in the direction of interest are brighter in the image.

FIGS. 2 and 3 show examples of rendering a TEE acquired heart volume of a patient for viewing a flow jet. The color information for flow is overlaid with a rendering of the tissue or B-mode information, or the B-mode and flow color are rendered together. Two planar images are included in each example on the left. Alpha blending is used for projection rendering the images on the right in each Figure. Both variance and velocity thresholds are applied in the rendering. The transparency is set based on the flow direction in FIG. 3. In FIG. 2, the transparency is unity or not adjusted based on the flow direction. In FIG. 2, a red region is shown between two blue regions. The arrow points to the lower of the two blue regions. Blue represents velocities moving away from the transducer. Red represents velocities moving towards the transducer. The lower blue region in FIG. 2 covers or interferes with part of the red region, so the flow moving away from the transducer obscures the deeper flow moving towards the transducer. By applying the transparency based on the direction of the flow, the blue regions become blurry or more transparent in the rendering, as shown in FIG. 3. The blocked portion of the red region or flow towards the transducer is now visible due to the transparency being set as a function of direction.

In another embodiment, vector flow visualization is generated as a two-dimensional image or as a volume color flow image. Vector color flow indicates the direction of flow in the image by utilizing different color schemes, a field of arrows, moving particles, streaming lines, or other direction indicators. However, the added two or three-dimensional flow direction information may be both helpful and overwhelming to the users. To highlight the intended flow in the display, the flow of interest going in a certain range of directions is displayed or highlighted with a direction indicators while the direction indicators of flow going in other directions are made more transparent. The direction of flow is used to modulate the transparency of the vector flow display. Any function of the direction can be used.

FIG. 4 shows a system 10 for transparency control in ultrasound medical imaging. The system 10 includes a transducer 12, an ultrasound imaging system 18, a processor 20, a memory 22, and a display 24. Additional, different, or fewer components may be provided. For example, the system 10 includes a user interface for changing thresholds, clutter filtering, direction selection, amount of transparency change and/or transparency values to use. In one embodiment, the system 10 is a medical diagnostic ultrasound imaging system. In other embodiments, the processor 20 and/or memory 22 are part of a workstation or computer different or separate from the ultrasound imaging system 18. The workstation is adjacent to or remote from the ultrasound imaging system 18.

The system 10 implements the method of FIG. 1, such as to render an image as shown in FIG. 3. One or more, such as all of the acts, of FIG. 1 are performed by the system 10. For example, the transducer 12 and ultrasound imaging system 18 are used to acquire data. The processor 20 and/or the ultrasound imaging system 18 determine the direction of flow for different voxels and set the transparency based on the flow. The ultrasound imaging system 18 and/or the processor 20 volume render the acquired data set using the direction-based transparency. The rendered image is displayed on the display 24.

The transducer 12 is a single element transducer, a linear array, a curved linear array, a phased array, a 1.5 dimensional array, a two-dimensional array, a radial array, an annular array, a multidimensional array, a wobbler, or other now known or later developed array of elements. The elements are piezoelectric or capacitive materials or structures. In one embodiment, the transducer 12 is adapted for use external to the patient, such as including a hand held housing or a housing for mounting to an external structure. More than one array may be provided, such as a support arm for positioning two or more (e.g., four) wobbler transducers adjacent to a patient (e.g., adjacent an abdomen of a pregnant female). The wobblers mechanically and electrically scan and are synchronized to scan the patient and form a composite volume. In other embodiments, a single hand-held transducer is provided for scanning different planes while being moved or for scanning a volume from one or more acoustic windows. In alternative embodiments, the transducer 12 is adapted for use within the patient, such as being on a transesophageal or cardiac catheter probe.

The transducer 12 converts between electrical signals and acoustic energy for scanning a region of the patient body. The region of the body scanned is a function of the type of transducer array and position of the transducer 12 relative to the patient. For example, a linear transducer array may scan a rectangular or square, planar region of the body. As another example, a curved linear array may scan a pie shaped region of the body. Scans conforming to other geometrical regions or shapes within the body may be used, such as Vector® scans. The scans are of a two-dimensional plane. Different planes may be scanned by moving the transducer 12, such as by rotation, rocking, and/or translation. A volume is scanned. The volume may be scanned by electronic steering alone (e.g., volume scan with a two-dimensional array), or mechanical and electrical steering (e.g., a wobbler array or movement of an array for planar scanning to scan different planes).

The ultrasound imaging system 18 is a medical diagnostic ultrasound system. For example, the ultrasound imaging system 18 includes a transmit beamformer, a receive beamformer, a detector (e.g., B-mode and Doppler), a scan converter, and the display 24 or a different display. The ultrasound imaging system 18 connects with the transducer 12, such as through a releasable connector. Transmit signals are generated and provided to the transducer 12. Responsive electrical signals are received from the transducer 12 and processed by the ultrasound imaging system 18.

In one embodiment, the ultrasound imaging system 18 includes a flow estimator. A wall or clutter filter and corner turning memory may be provided. The filter reduces the influence of data from tissue while maintaining velocity information from fluids or alternatively reduces the influence of data from fluids while maintaining velocity information from tissue. The filter has a set response or may be programmed, such as altering operation as a function of signal feedback or other adaptive process.

The flow estimator is a Doppler processor or cross-correlation processor for estimating the flow data. In alternative embodiments, another device now known or later developed for estimating velocity, energy, and/or variance from any or various input samples may be provided. The flow estimator receives a plurality of samples associated with a substantially same location at different times and estimates a Doppler shift frequency, based on a change or an average change in phase between consecutive signals from the same location. Velocity is calculated from the Doppler shift frequency. Alternatively, the Doppler shift frequency is used as a velocity. The energy and variance may also be calculated.

Flow data (e.g., velocity, energy, or variance) is estimated for spatial locations in the scan volume from the beamformed samples. For example, the flow data represents a plurality of different planes in the volume. An estimate of flow is provided for each voxel associated with flow in a volume.

The flow estimator may apply one or more thresholds to identify sufficient motion information. For example, velocity and/or energy thresholding for identifying velocities is used. In alternative embodiments, a separate processor or filter applies thresholds. The flow estimator outputs ultrasound flow data for the volume.

The ultrasound imaging system 18 is configured by user settings or selection of a scan application to cause a scan of an internal region of a patient with the transducer 12 and generate data representing the region as a function of the scanning. The scanned region is adjacent to the transducer 12. For example, the transducer 12 is placed against an abdomen or within a patient. Ultrasound data is acquired and used to estimate the ultrasound flow data, such as velocity or energy data. The ultrasound flow data may be filtered, thresholded, scan converted, and/or interpolated to a three-dimensional grid for rendering. The ultrasound flow data anywhere along the processing path represents flow within the volume of the patient.

In another embodiment, the ultrasound imaging system 18 is a workstation or computer for processing ultrasound data. Ultrasound flow data is acquired using an imaging system connected with the transducer 12 or using an integrated transducer 12 and imaging system. The data at any level of processing (e.g., radio frequency data (e.g., I/O data), beamformed data, estimated data, and/or scan converted data) is output or stored. For example, the data is output to a data archival system or output on a network to an adjacent or remote workstation. The ultrasound imaging system 18 processes the data further for analysis, diagnosis, and/or display.

The processor 20 is one or more general processors, digital signal processors, application specific integrated circuits, field programmable gate arrays, controllers, analog circuits, digital circuits, server, graphics processing units, graphics processors, combinations thereof, network, or other logic devices for rendering. A single device is used, but parallel or sequential distributed processing may be used. The processor 20 is part of the imaging system 18 or may be separate, such as in a separate computer or workstation local to or spaced from the imaging system 18.

The processor 20 is configured by software and/or hardware to render. The processor implements the determination, setting, and rendering acts 42, 44, and 46 discussed above or different acts. For example, the processor 20 determines the direction of flow for various voxels, sets the transparency based on direction of flow, and renders using the transparencies.

In one embodiment, the processor 20 modulates transparencies of data responsive to the scan as a function of direction of flow. One or multi-dimensional (two or three) flow direction information may be used. For one dimensional flow, one direction is mapped to one transparency level (or amount of transparency change) and another direction is mapped to different transparency level (or amount of transparency change). For multi-directional flow, a group of directions are mapped to one transparency level or amount of change and another group of directions are mapped to another transparency level or amount of change. Alternatively, more than two transparency levels or amounts of change are provided, such as transparency different for each direction or for different ranges of directions.

The transparency that otherwise would be used in rendering is modulated by the direction of flow. The processor 20 determines the transparency using direction information and/or alters transparency using the direction information. Any adjustment may be made, such as scaling the opacity by multiplying by a weight. The transparencies are modulated by the processor 20 such that transparency is increased for one direction and decreased for an opposite direction. The processor 20 adjusts the transparency for each of a plurality of volume positions or voxels.

The processor 20 is configured to render the image from the medical data, such as from a combination of B-mode and ultrasound flow data (e.g., render a velocity or energy image with color modulated as a function of magnitude). Any type of rendering may be provided, such as surface rendering or volume rendering (e.g., projection rendering). For projection rendering, compositing is performed along a plurality of ray lines. The compositing for each ray line continues until saturation of the accumulated opacity or the end of the volume. The depth of the data along each of the ray lines contributing to the compositing is a function of the opacity. For example, adjusting of the transparency increases the depth of the contribution along at least one of the ray lines. By making intervening flow structures more transparent, with or without saturation, deeper flow structures may be more visible in the rendered image. The transparencies are used in rendering to highlight a flow region relative to another based on direction of flow.

The memory 22 is a tape, magnetic, optical, hard drive, RAM, buffer or other memory. The memory 22 stores the ultrasound flow data from one or more scans, at different stages of processing, and/or as a rendered image. Direction information and/or transparency settings may be stored.

The memory 22 is additionally or alternatively a non-transitory computer readable storage medium with processing instructions. Data representing instructions executable by the programmed processor 20 is provided for transparency control in three-dimensional rendering or other flow imaging. The instructions for implementing the processes, methods and/or techniques discussed herein are provided on non-transitory computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The display 24 is a CRT, LCD, projector, plasma, printer, or other display for displaying two-dimensional images or three-dimensional representations or renderings. The display 24 generates a flow image of the three-dimensional rendering, such as shown in FIG. 3. The image data is provided to the display 24. The display 24 displays the rendered image from the provided image data. The image represents a view from viewer's perspective of the internal volume of the patient. Data from different locations is represented in the image. The color brightness reflects the flow data and transparency. The colors in the image for one direction are brighter than or more visible than colors in the image for a different direction. The image is a function of the transparency adjusted as the function of the direction of flow. The image on the display 24 is output from volume or surface rendering.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for transparency control in medical ultrasound flow imaging, the method comprising:
acquiring ultrasound flow data as voxels representing a volume of a patient;
determining, by a processor, a direction of flow for each voxel;
setting, by the processor, a transparency for each voxel as a function of the respective direction of the flow; and
rendering, by the processor, an image of the volume of the patient with the ultrasound flow data, the rendering being a function of the transparencies for the voxels.

2. The method of claim 1 wherein acquiring the ultrasound flow data comprises acquiring color Doppler data.

3. The method of claim 1 wherein acquiring the ultrasound flow data comprises acquiring velocity estimates for the voxel.

4. The method of claim 1 wherein determining the direction comprises determining a sign of velocity for the voxel.

5. The method of claim 1 wherein determining the direction comprises determining a direction of flow to and away from a transducer.

6. The method of claim 1 wherein determining the direction comprises determining a two or three-dimensional direction.

7. The method of claim 1 wherein setting comprises setting the transparency to be greater for voxels with a first direction and to be less for a voxels with a second direction different than the first direction.

8. The method of claim 1 wherein setting comprises setting the transparency to be greater where the direction is towards a transducer and to be lesser where the direction is away from the transducer.

9. The method of claim 1 wherein rendering comprises rendering the image as a color Doppler image of valvular flow in a heart of the patient.

10. The method of claim 1 wherein rendering comprises weighting the ultrasound flow data of the voxels as a function of the respective transparencies and projection rendering along ray lines through the voxels using the weighted ultrasound flow data.

11. The method of claim 1 wherein acquiring comprises acquiring the ultrasound flow data as three-dimensional energy, velocity and/or variance data, wherein determining the direction comprises determining the direction of velocities along scan lines, and wherein rendering comprises rendering the image as an energy, velocity, and/or variance image.

12. A system for transparency control in three-dimensional ultrasound imaging, the system comprising:
a transducer;
an ultrasound imaging system configured to scan an internal volume of a patient with the transducer;
a processor configured to modulate transparencies of data responsive to the scan as a function of direction of flow and three-dimensionally render a flow image from the data as a function of the transparencies; and
a display operable to display the flow image of the internal volume, the flow image comprising a color image where colors associated with one direction are more opaque than colors associated with another direction.

13. The system of claim 12 wherein the ultrasound imaging system comprises a Doppler processor configured to estimate velocity or energy values for each of a plurality of voxels representing the internal volume, wherein the directions of flow comprises direction of velocity flow, and wherein the flow image comprises a velocity or energy image with color modulated as a function of magnitude of the velocity or energy.

14. The system of claim 12 wherein the processor is configured to modulate the transparencies such that transparency is increased for one direction and decreased for an opposite direction.

15. The system of claim 12 wherein the processor is configured to modulate the transparencies using a one-dimensional flow.

16. The system of claim 12 wherein the processor is configured to modulate the transparencies using a multi-dimensional flow direction.

17. In a non-transitory computer readable storage medium having stored therein data representing instructions executable by a programmed processor for transparency control in ultrasound imaging, the storage medium comprising instructions for:
performing color Doppler imaging; and
setting opacity for the performing of the color Doppler imaging as a function of a direction of flow.

18. The non-transitory computer readable storage medium of claim 17 wherein performing the color Doppler imaging comprises volume rendering a Doppler velocity image, and wherein setting the opacity comprises setting opacities of voxels representing a volume as a function of the direction of the flow of velocities.

19. The non-transitory computer readable storage medium of claim 17 wherein performing the color Doppler imaging comprises generating a vector flow display, and wherein setting the opacity comprises setting the opacities as a function of the direction of the flow velocities for different locations.

20. The non-transitory computer readable storage medium of claim 17 wherein setting comprises increasing opacity for flow towards a transducer and decreasing opacity for flow away from the transducer.
